(19) United States
(12) Patent Application Publication
(10) Pub. No.: US 2013/0345096 A1
(43) Pub. Date: Dec. 26, 2013

(54) Microfluidic Chip Automatic System With Optical Platform

(71) Applicant: Chin-Feng Wan, Hsinchu City (TW)

(72) Inventor: Chin-Feng Wan, Hsinchu City (TW)

(21) Appl. No.: 13/925,369

(22) Filed: Jun. 24, 2013

(30) Foreign Application Priority Data
Jun. 25, 2012 (TW) 101212200

(51) Int. Cl. G01N 33/50

(52) U.S. Cl. CPC G01N 33/50
USPC 506/37

(57) ABSTRACT

A microfluidic chip automatic system includes a microfluidic chip platform and an optical platform. The microfluidic chip platform includes a microfluidic chip, a fluid source, a gas source, and a controller. A time sequence of charging a high pressure gas from the gas source into the microfluidic chip and discharging the high pressure gas from the microfluidic chip is controlled by the controller through plural solenoid valves. The optical platform includes a light source, plural lenses, a digital micromirror device, a grating device and a reflective mirror. A light beam provided by the light source is guided to the microfluidic chip. The digital micromirror device includes plural micromirrors. The optical switching states of the micromirrors are controlled by a computer, so that a position of the microfluidic chip to carry out a photochemical reaction is correspondingly controlled.

### FIELD OF THE INVENTION

The present invention relates to a microfluidic chip automatic system, and more particularly to a microfluidic chip automatic system with an optical platform.

### BACKGROUND OF THE INVENTION

A biochip is a miniaturized device that allows specific biochemical reactions between specified biological materials (e.g. nucleic acid or protein) and other under-test biological samples by employing a microelectromechanical (MEMS) technology. After the reaction signals are quantified by various sensors, the possible biochemical reactions can be realized. In other words, the miniaturized device fabricated by a microelectromechanical technology and a biological technology is referred as the biochip. For example, the biochip is a microfluidic chip or a lab-on-a-chip. The applications of the biochip cover the disease diagnosis, the gene probe, the pharmaceutical technology, the microelectronic technology, the semiconductor technology, the computer technology, and the like.

Recently, due to the rapid development of biomedicine and the rising awareness of personal health, the demands on fast symptom detection and correct diagnosis are gradually increased. The medical organizations or research organizations pay much attention on seeking the platform for automatically and quickly acquire large numbers of detection data. With the development and maturity of the microelectromechanical technology, the microfluidic chip becomes a rapidly developing research field. By means of the microelectromechanical technology, a series of steps of carrying out the complicated biological reaction (e.g. sampling, sample handling, sample separation, reagent reaction and detection) can be integrated into a small microfluidic chip. In other words, the microfluidic chip has many benefits such as low cost, rapid detection and low reagent and sample consumption. Therefore, there is a need of providing a microfluidic chip automatic system.

Regardless of the synthesis stages or the detection stages of the biochips, the photochemical reaction plays an important role. In other words, the integration of an optical path system of the photochemical reaction into the microfluidic chip automatic system is an important subject of the present invention.

### SUMMARY OF THE INVENTION

The present invention provides a microfluidic chip automatic system with an optical platform in order for automatically detecting biological molecule, accelerating the detecting process and detecting a large number of different samples.

The present invention also provides a microfluidic chip automatic system with an optical platform. The microfluidic chip automatic system is used for performing an optical imaging operation according to a predetermined pattern of a digital micromirror device of the optical platform. By guiding a light beam to a microfluidic chip on a sample platform, the position of carrying out the photochemical reaction on the sample platform can be effectively controlled.

In accordance with an aspect of the present invention, there is provided a microfluidic chip automatic system. The microfluidic chip automatic system includes a microfluidic chip platform and an optical platform. The microfluidic chip platform includes a microfluidic chip, a fluid source, a gas source, and a controller. The microfluidic chip includes a base layer, a fluid layer and a gas regulating layer. The base layer includes a microarray reaction zone. The fluid layer is disposed over the base layer, and includes plural flow channels for introducing and collecting a reagent. The gas regulating layer is disposed over the fluid layer for controlling open/close states of the flow channels, thereby controlling a flowing condition of a fluid in the fluid layer. The fluid source includes the reagent, which is introduced into the fluid layer of the microfluidic chip. The gas source provides a high pressure gas to the gas regulating layer of the microfluidic chip. The controller is connected with the gas source, and includes plural solenoid valves. A time sequence of charging the high pressure gas from the gas source into the microfluidic chip and discharging the high pressure gas from the microfluidic chip is controlled by the controller through the plural solenoid valves. The optical platform includes a light source, plural lenses, a digital micromirror device, a grating device and a reflective mirror. A light beam provided by the light source is guided to the microfluidic chip of the microfluidic chip platform. The digital micromirror device includes plural micromirrors. The optical switching states of the micromirrors are controlled by a computer, so that a position of the microfluidic chip to carry out a photochemical reaction is correspondingly controlled.

The above contents of the present invention will become more readily apparent to those ordinarily skilled in the art after reviewing the following detailed description and accompanying drawings, in which:

### BRIEF DESCRIPTION OF THE DRAWINGS

FIG. 1 schematically illustrates the architecture of a microfluidic chip automatic system according to an embodiment of the present invention;

FIG. 2 is a schematic exploded view illustrating the structure of a microfluidic chip according to an embodiment of the present invention;

FIG. 3 schematically illustrates the relationships between the fluid layer and the gas regulating layer of the microfluidic chip of FIG. 2, in which the gas regulating layer is disposed over the fluid layer;

FIG. 4 schematically illustrates the architecture of the controller of the microfluidic chip used in the microfluidic chip automatic system according to an embodiment of the present invention;

FIG. 5 schematically illustrates the execution of the solenoid valve control program used in the microfluidic chip automatic system according to an embodiment of the present invention; and

FIG. 6 schematically illustrates a digital micromirror device used in the microfluidic chip automatic system according to an embodiment of the present invention.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

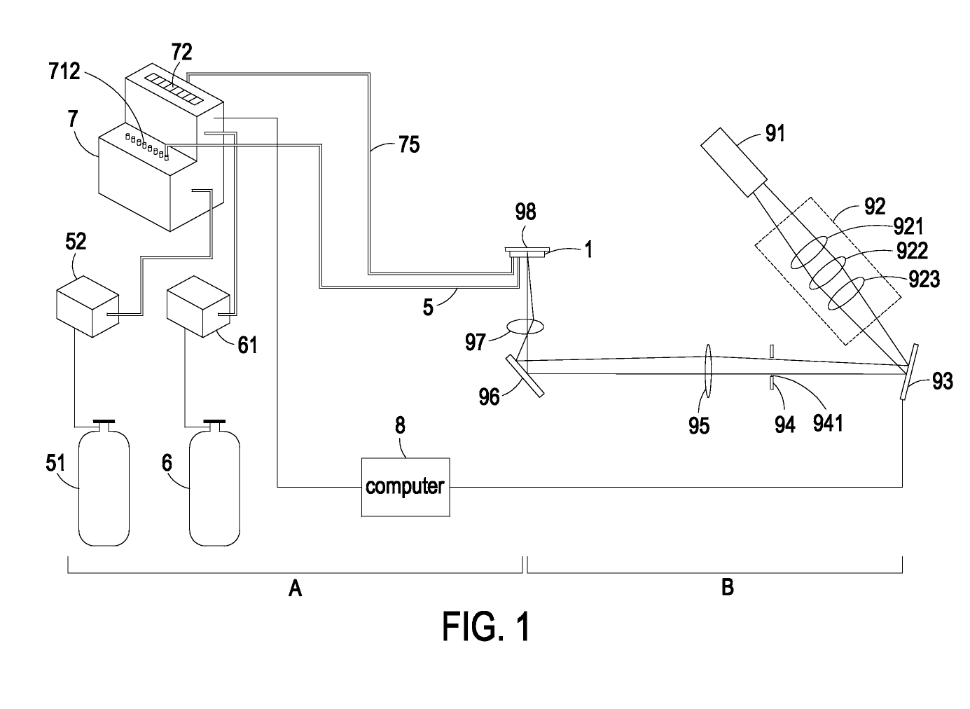

FIG. 1 schematically illustrates the architecture of a microfluidic chip automatic system according to an embodiment of the present invention. As shown in FIG. 1, the microfluidic chip automatic system comprises a microfluidic chip platform A and an optical platform B. The detailed structures and the layout configurations of the microfluidic chip platform A and the optical platform B will be illustrated as follows.

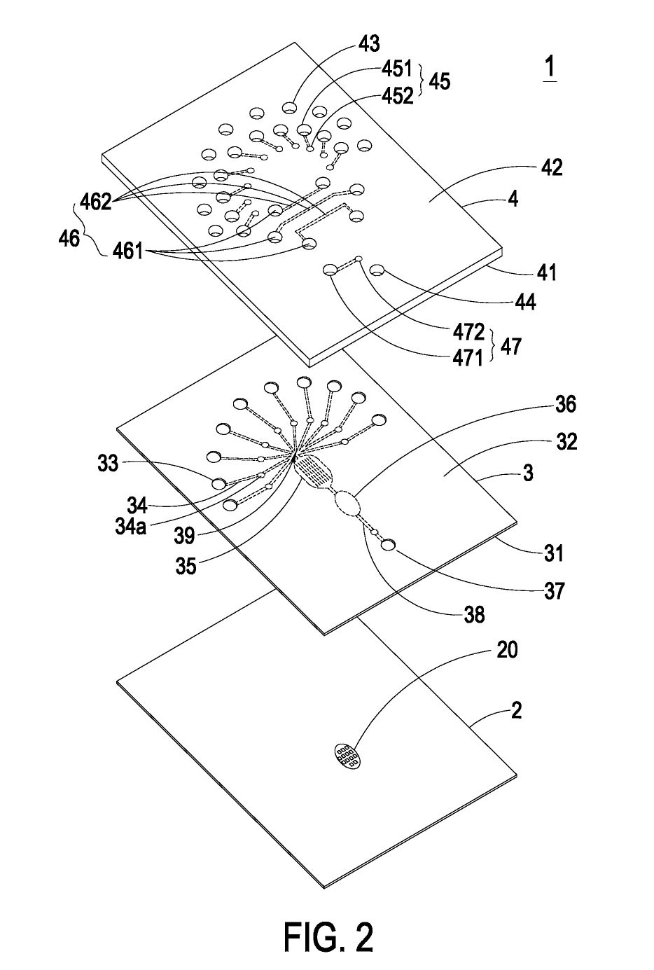

FIG. 2 is a schematic exploded view illustrating the structure of a microfluidic chip according to an embodiment of the present invention. As shown in FIG. 2, the microfluidic chip 1 comprises a base layer 2, a fluid layer 3, and a gas regulating layer 4. The base layer 2 has a microarray reaction zone 20. The fluid layer 3 is disposed over the base layer 2 to cover the base layer 2. The fluid layer 3 has flow channels, wherein samples and detecting reagents may be introduced into or collected in the flow channels. The gas regulating layer 4 is disposed over the fluid layer 3 to cover the fluid layer 3. The gas regulating layer 4 is used for controlling the open/close states of the flow channels in order to control the flowing condition of the fluid in the fluid layer 3.

The fluid layer 3 is made of polydimethyl siloxane (PDMS). The fluid layer 3 has a first surface 31 facing the base layer 2 and a second surface 32 facing the gas regulating layer 4. Moreover, the fluid layer 3 comprises plural solution inlets 33, plural micro channels 34, a buffer region 39, a diffluent region 35, a reactive region 36, and a solution outlet 37. The plural solution inlets 33 are formed in the second surface 32 of the fluid layer 3. The samples, reagents and washing solutions may be introduced into the fluid layer 3 through different solution inlets 33. The plural micro channels 34 are concavely formed in the first surface 31 of the fluid layer 3. In addition, the plural micro channels 34 are in communication with and arranged between the plural solution inlets 33 and the buffer region 39. The buffer region 39, the diffluent region 35 and the reactive region 36 are also concavely formed in the first surface 31 of the fluid layer 3. In addition, the buffer region 39 and the diffluent region 35 are in communication with each other. In order to mix the samples with the reagents, the mixed fluid is collected and mixed in the diffluent region 35. The reactive region 36 is in communication with the diffluent region 35, and aligned with the microarray reaction zone 20 of the base layer 2. The specific reaction between the under-test molecule of the sample and a probe molecule (not shown) occurs at the microarray reaction zone 20, so that the under-test molecule can be detected. Furthermore, the solution outlet 37 is formed in the second surface 32 of the fluid layer 3. The waste solution produced by the specific reaction is exhausted out from the solution outlet 37.

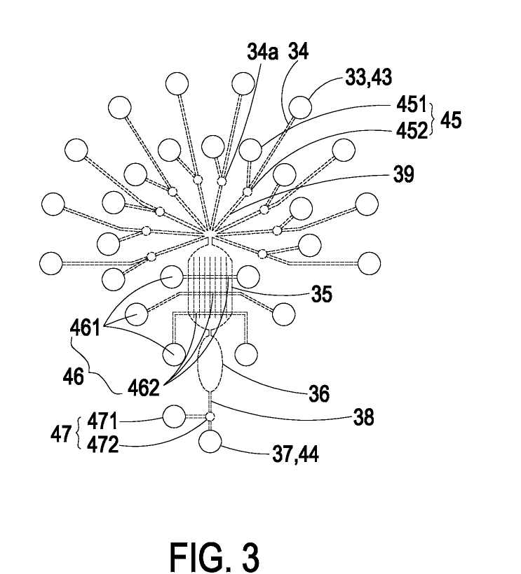

FIG. 3 schematically illustrates the relationships between the fluid layer and the gas regulating layer of the microfluidic chip of FIG. 2, in which the gas regulating layer is disposed over the fluid layer. Please refer to FIGS. 2 and 3. In this embodiment, the gas regulating layer 4 is made of polydimethyl siloxane (PDMS). The gas regulating layer 4 comprises a first surface 41 and a second surface 42, wherein the first surface 41 faces the fluid layer 3 and the second surface 42 is opposed to the first surface 41. Moreover, the gas regulating layer 4 comprises plural first slots 43, a second slot 44, plural micro valves 45, and a micropump group 46. The first slots 43 are aligned with respective solution inlets 33 of the fluid layer 3 and in communication with respective solution inlets 33. The second slot 44 is aligned with the solution outlet 37 of the fluid layer 3 and in communication with the solution outlet 37. The plural micro valves 45 may be driven by gases (or a small amount of water), so that the circular membranes 34a of the micro channels 34 are selectively blocked or unblocked. The micropump group 46 may be driven to allow the fluid within the micro channels 34 to be flowed in the direction toward the reactive region 36.

Moreover, each of the plural micro valves 45 is aligned with a corresponding micro channel 34. Each of the plural micro valves 45 comprises a valve pore 451 and a valve chamber 452. The valve pore 451 is formed in the second surface 42 of the gas regulating layer 4. The valve chamber 452 is concavely formed in the first surface 41 of the gas regulating layer 4 and disposed over the corresponding circular membranes 34a of the micro channel 34. The valve pore 451 is connected with a silicone tube and a solenoid valve (not shown). Consequently, a gas may be introduced into the valve chamber 452 through the silicone tube and the valve pore 451. The gas may force the fluid layer 3 underlying the valve chamber 452 to be moved downwardly, so that the circular membranes 34a of the micro channel 34 is compressed to block the fluid within the micro channel 34. In other words, the micro valve 45 is opened or closed by selectively charging the gas into the valve chamber 452 or discharging the gas from the valve chamber 452. On the other hand, once the gas is discharged, the compressed fluid layer 3 is moved upwardly and returned to the original position. Consequently, a negative pressure is generated to facilitate the fluid to flow within the micro channel 34.

Please refer to FIG. 2 again. In this embodiment, the gas regulating layer 4 has a micropump group 46. The micropump group 46 comprises at least three pump pores 461 and at least three pump chambers 462. The pump pores 461 are formed in the second surface 42 of the gas regulating layer 4. The pump chambers 462 are in communication with corresponding pump pores 461. Moreover, the pump chambers 462 are concavely formed in the first surface 41 of the gas regulating layer 4 and disposed over the diffluent region 35. Moreover, each of the pump pores 461 is connected with a silicone tube and a solenoid valve (not shown). Consequently, a gas may be introduced into the corresponding pump chamber 462 through the silicone tube and the pump pore 461. The gas may force the fluid layer 3 underlying the pump chamber 462 to be moved downwardly, so that the diffluent region 35 is compressed to block the fluid within the diffluent region 35. The three pump chambers 462 of the micropump group 46 are disposed over different segments of the diffluent region 35. By sequentially and alternately charging the gas into the pump chamber 462 and discharging the gas from the pump chamber 462, the three pump chambers 462 and the diffluent region 35 may cooperate to produce a peristaltic pumping action. Due to the peristaltic pumping action, the fluid is continuously pushed to the reactive region 36, so that the biological detecting reaction is performed at the reactive region 36.

Moreover, the fluid layer 3 further comprises a liquid collecting channel 38. The liquid collecting channel 38 is concavely formed in the first surface 31 of the fluid layer 3. Moreover, the liquid collecting channel 38 is in communication with and arranged between the reactive region 36 and the solution outlet 37. Moreover, the gas regulating layer 4 further comprises a liquid collecting valve 47. The liquid collecting valve 47 is aligned with the collecting channel 38. The liquid collecting valve 47 comprises a valve pore 471 and a valve chamber 472. The valve pore 471 is formed in the second surface 42 of the gas regulating layer 4. The valve chamber 472 is concavely formed in the first surface 41 of the gas regulating layer 4, and disposed over the liquid collecting channel 38. The valve pore 471 is connected with a silicone tube and a solenoid valve (not shown). Consequently, a gas may be introduced into the valve chamber 472 through the silicone tube and the valve pore 471. The gas may force the fluid layer 3 underlying the valve chamber 472 to be moved downwardly, so that the liquid collecting channel 38 is blocked. On the other hand, once the gas is discharged, the compressed fluid layer 3 is moved upwardly and returned to the original position. Meanwhile, a negative pressure is generated to facilitate the fluid to flow to the solution outlet 37 through the liquid collecting channel 38, and thus waste solution produced by the specific reaction is exhausted out from the solution outlet 37. In other words, the liquid collecting valve 47 is opened or closed by selectively charging the gas into the valve chamber 472 or discharging the gas from the valve chamber 472.

In an embodiment, the thickness of the fluid layer 3 is about 42 μm, the depth of the micro channel 34 is about 10 μm˜18 μm, the thickness of the gas regulating layer 4 is about 4 mm, and the depths of the valve chambers 452, 472 and the pump chambers 462 are about 100 μm. The above dimensions are not restricted. It is noted that the numbers and arrangements of the solution inlets 33, the micro channels 34, the second slot 44 and the micropump group 46 may be varied according to the practical requirements.

Please refer to FIG. 1 again. The microfluidic chip platform A principally comprises a microfluidic chip 1, a fluid source, a gas source 6, a controller 7, and a computer 8. The microfluidic chip 1 is placed on a sample platform 98. The sample platform 98 may be placed on a microscope device (not shown). Via the microscope device, the reaction of the microfluidic chip 1 can be observed by the user. The fluid source comprises samples, reagents and washing solutions. The samples, the reagents and the washing solutions are introduced into corresponding first slots 43 of the microfluidic chip 1 through fluid pipes 5, wherein the first slots 43 are aligned with respective solution inlets 33. The gas source 6 is a cylinder containing a high pressure gas (e.g. nitrogen gas) or an air compressor. The gas source 6 is used for providing the high pressure gas to the microfluidic chip 1. A flowmeter 61 is connected with the gas source 6 for controlling the output flow rate of the gas source 6. The controller 7 is connected with the gas source 6 for controlling the time sequence of charging the high pressure gas from gas source 6 into the microfluidic chip 1 and discharging the high pressure gas from the microfluidic chip 1 through solenoid valves 72. The high pressure gas is introduced into the valve pores 451, 471 and the pump pores 461 through gas pipes 75. The computer 8 is connected with the controller 7 for controlling on/off states of the solenoid valves 72 through a solenoid valve control program.

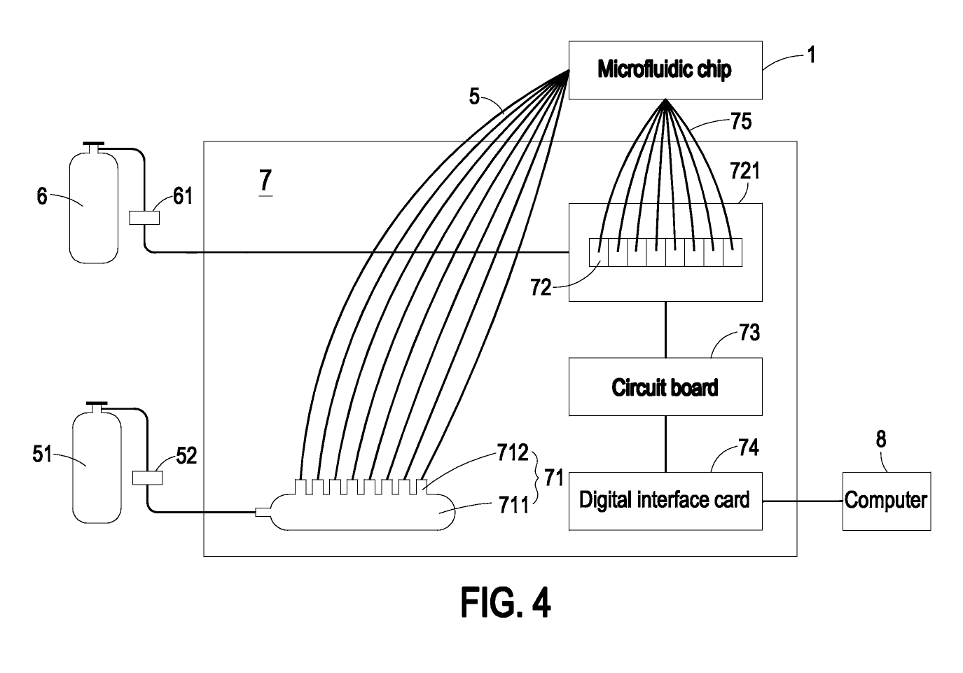

FIG. 4 schematically illustrates the architecture of the controller of the microfluidic chip used in the microfluidic chip automatic system according to an embodiment of the present invention. As shown in FIG. 4, the controller 7 comprises a manifold device 71, plural solenoid valves 72, a circuit board 73, and a digital interface card 74. The manifold device 71 comprises a main body 711 and plural outlets 712. The main body 711 is in communication with the plural outlets 712. The plural outlets 712 are connected with corresponding fluid pipes 5. After the reagents and the washing solutions of the fluid source are introduced into corresponding fluid pipes, the first ends of the fluid pipes are connected with corresponding outlets 712 of the manifold device 71, and the second ends of the fluid pipes are connected with corresponding inlets of the microfluidic chip 1. For providing a pushing force to the fluids within the fluid pipes 5, the manifold device 71 may be independently connected with an additional gas source 51. Similarly, the gas source 51 is a cylinder containing a high pressure gas (e.g. nitrogen gas) or an air compressor. A flowmeter 52 is used for controlling the output flow rate of the gas source 51. Optionally, after the high pressure gas from the gas source 51 reaches the equilibrium state in the main body 711 of the manifold device 71, the high pressure gas is uniformly outputted from the outlets 712. Under this circumstance, the high pressure gas in each fluid pipe 5 has the identical flow rate and is substantially in the equilibrium state. The plural solenoid valves 72 are disposed on a fixing seat 721. The fixing seat 721 is connected with the gas source 6. Moreover, the plural solenoid valves 72 are connected with first ends of respective gas pipes 75. The second ends of the gas pipes 75 are connected with the corresponding pores 451, 471 and 461 of the microfluidic chip 1. Moreover, the plural solenoid valves 72 are connected with the circuit board 73. The circuit board 7 is further connected with the digital interface card 74. The digital interface card 74 is further connected with the computer 8. The on/off states of the solenoid valves 72 are driven by the computer 8 in order to control the time sequence of introducing the high pressure gas into corresponding pores of the microfluidic chip 1 through the gas pipes 75.

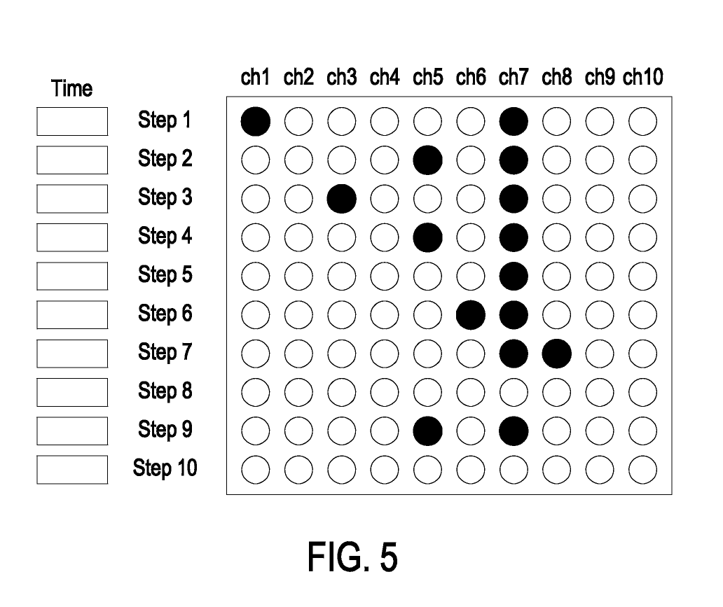

In an embodiment, the solenoid valve 72 is a 3 port solenoid valve. The on/off states of the solenoid valves 72 are controlled by a solenoid valve control program (e.g. Lab View software). By the solenoid valve 72, an electronic potential energy which is digitally inputted into or outputted from a timing interface card may be converted into different gas pressure levels (e.g. 0˜0.15 MPa). FIG. 5 schematically illustrates the execution of the solenoid valve control program used in the microfluidic chip automatic system according to an embodiment of the present invention. As shown in FIG. 5, the horizontal axis indicates the flow channels that are controlled by the solenoid valves. The vertical axis indicates the time sequence of the controlling steps. The contents of the blank grids are the time points that are written by the user. The solid circle indicates the on state of the solenoid valve. The dotted circle indicates the on state of the solenoid valve. By using the computer 8 to perform automatic control, many steps may be programmed to automatically introduce the reactive samples, the reagents and the washing solutions. When the high pressure gas is introduced into the chambers 452, 462 and 472 of the gas regulating layer 4, the gas may force the underlying fluid layer 3 to be moved downwardly, so that the fluid within the flow channels is blocked. In other words, by charging the gas into the chambers 452, 462 and 472 or discharging the gas from the chambers 452, 462 and 472, the flow channels are selectively opened or closed, and the desired volume of the liquid can be controlled. As a consequence, the microfluidic chip automatic system of the present invention may be used to perform the parallel multitasking analysis of multiple reagents and implement the multi-step biochemical reactions.

Please refer to FIG. 1 again. The optical platform B is a maskless lithography optical platform. In this embodiment, the optical platform B comprises a light source 91, a first lens group 92, a digital micromirror device (DMD) 93, a grating device 94, a second lens 95, a reflective mirror 96, and a third lens 97. The optical platform B is used for performing an optical imaging operation according to a predetermined pattern of the digital micromirror device 93. By guiding a light beam to the microfluidic chip 1 on the sample platform 98, the position of carrying out the photochemical reaction on the microfluidic chip 1 can be effectively controlled.

The light source 91 is used for providing a light beam. An example of the light source 91 includes but is not limited to a high pressure mercury lamp. In case that the light source 91 is a high pressure mercury lamp, the light beam is a UV light beam. The first lens group 92 is arranged between the light source 91 and the digital micromirror device 93 for guiding the light beam from the light source to the digital micromirror device 93. Moreover, the first lens group 92 comprises at least two lenses. In this embodiment, the first lens group 92 comprises three lenses 921, 922 and 923. After the curvatures of these lenses are precisely calculated according to the imaging requirements, the efficacy of guiding the light beam is enhanced. In an embodiment, the three lenses 921, 922 and 923 are all plano-convex lenses. Alternatively, in some other embodiments, the three lenses 921, 922 and 923 are all biconvex lenses. Alternatively, in some other embodiments, the three lenses 921, 922 and 923 may be selected from the combination of biconvex lenses and plano-convex lenses.

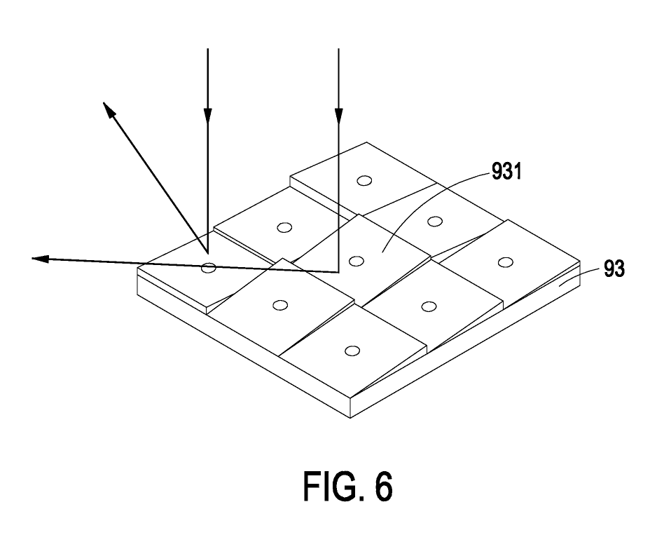

The digital micromirror device 93 comprises plural micromirrors 931 (see FIG. 6). These micromirrors 931 are arranged in an array with a desired size. The optical switching states of the micromirrors 931 are controlled by the computer 8, so that a patterned light beam is outputted from the digital micromirror device 93. In an embodiment, the computer 8 is used for converting a designed image into a control signal and adjusting the orientation of the micromirrors 931, thereby controlling the optical switching states of the micromirrors 931. That is, since the optical switching states of respective micromirrors 931 are controlled by the computer 8, the light beam is selectively to be guided to be directed toward the grating device 94 or away from the grating device 94. Since the operations of the plural micromirrors 931 are controlled by the computer 8 according to the desired image, the light beam provided by the light source 91 is converted into the patterned light beam, and the patterned light beam is directed to the grating device 94.

The grating device 94 comprises an adjustable grating window 941 for allowing a portion of the patterned light beam to go through. Since the size of the grating window 941 is adjustable, the light amount to be introduced into the grating window 941 can be controlled in order to increase the light contrast and the resolution of the image. Of course, the size of the grating window 941 may be adjusted according to the practical requirements.

After the patterned light beam is transmitted through the grating window 941 of the grating device 94, the patterned light beam is directed to the second lens 95. By the second lens 95, the patterned light beam is guided to the reflective mirror 96. The reflective mirror 96 is used for changing the path of the patterned light beam, so that the patterned light beam is directed in a direction toward the sample platform 18. Then, the patterned light beam is directed to the sample platform 18 through the third lens 97. In an embodiment, the third lens 97 is a focusing lens.

By integrating the microfluidic chip platform A with the optical platform B, the microfluidic chip automatic system of the present invention may be applied to the fabrication of a biochip. For example, for defining a microarray structure in the biochip, it is necessary to form a photoresist pattern layer on a substrate of a chip. Firstly, a photoresist layer (e.g. an epoxy-based photoresist material layer such as a SU-8 photoresist layer) is formed on a surface of the substrate. Then, by using the optical platform B to irradiate a specified position of the photoresist layer, the photoresist layer is subjected to polymerization. After a developing solution is used to remove the unpolymerized photoresist layer, the photoresist pattern layer is fabricated. Then, biological materials (e.g. nucleic acid or protein) are bonded onto the photoresist pattern layer, so that the biochip is fabricated. Since the photoresist pattern layer is formed by the maskless lithography optical platform of the present invention, it is not necessary to use the conventional costly photomask. Moreover, since the photoresist pattern layer is produced by a maskless lithography process, each spot of the microarray structure has a diameter smaller than 300 μm and the fabricating process is simplified.

Moreover, the microfluidic chip automatic system of the present invention may be applied to the synthesis of DNA. After a DNA is irradiated to generate broken bonds and the protective groups at the 5′-end of the nucleotide are removed, the nucleotide molecules (e.g. A, T, C, G) to be linked are subjected to a synthesizing reaction. After the unreacted nucleotide molecules are washed off, the steps of irradiating, adding nucleotide molecules and washing are repeatedly done. Consequently, the DNA with a desired sequence is synthesized. By using the microfluidic chip platform A to control each reaction step and using the optical platform B to control the irradiating position, the linking position of the nucleotide molecules on the chip in each synthesizing step can be determined. Consequently, plural DNA molecules with different sequences may be synthesized on the chip in the same fabricating process. In such way, a DNA chip for screening disease or detecting biologic molecules is prepared.

From the above descriptions, the present invention provides a microfluidic chip automatic system. The microfluidic chip automatic system comprises a microfluidic chip platform and an optical platform. A solenoid valve control program is installed in a computer for controlling on/off states of plural solenoid valves, thereby further controlling the flowing condition of the fluid in a microfluidic chip. In other words, the microfluidic chip automatic system of the present invention is capable of automatically detecting biological molecules and precisely carrying out the photochemical reaction. Since a series of steps of carrying out the complicated biological reaction are integrated into a small-area microfluidic chip, the behaviors of liquid on the micro scale may facilitate control of molecular diffusion and interaction. In other words, the microfluidic chip has many benefits such as low cost, rapid detection and low reagent and sample consumption. Moreover, the microfluidic chip automatic system of the present invention is capable of accelerating the detecting process and detecting a large number of different samples. In other words, the microfluidic chip automatic system of the present invention is effective for fast symptom detection and correct diagnosis. Moreover, since the optical platform is integrated into the microfluidic chip automatic system, the microfluidic chip automatic system can be used to control the photochemical reaction so as to be applied to the fabrication of a biochip. For example, the microfluidic chip automatic system of the present invention may be used to form a photoresist pattern layer on a substrate of a chip or synthesize DNA. In other words, the microfluidic chip automatic system of the present invention has industrial applicability.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A microfluidic chip automatic system, comprising:
a microfluidic chip platform comprising:
a microfluidic chip comprising a base layer, a fluid layer and a gas regulating layer, wherein said base layer comprises a microarray reaction zone, wherein said fluid layer is disposed over said base layer, and comprises plural flow channels for introducing and collecting a reagent, wherein said gas regulating layer is disposed over said fluid layer for controlling open/close states of said flow channels, thereby controlling a flowing condition of a fluid in the fluid layer;
a fluid source comprising said reagent, which is introduced into said fluid layer of said microfluidic chip;
a gas source for providing a high pressure gas to said gas regulating layer of said microfluidic chip; and
a controller connected with said gas source, and comprising plural solenoid valves, wherein a time sequence of charging said high pressure gas from said gas source into said microfluidic chip and discharging said high pressure gas from said microfluidic chip is controlled by said controller through said plural solenoid valves; and
an optical platform comprising a light source, plural lenses, a digital micromirror device, a grating device and a reflective mirror, wherein a light beam provided by said light source is guided to said microfluidic chip of said microfluidic chip platform, wherein said digital micromirror device comprising plural micromirrors, wherein optical switching states of said micromirrors are controlled by a computer, so that a position of said microfluidic chip to carry out a photochemical reaction is correspondingly controlled.

2. The microfluidic chip automatic system according to claim 1, wherein a solenoid valve control program is installed in said computer for controlling on/off states of said plural solenoid valves.

3. The microfluidic chip automatic system according to claim 1, further comprising a microscope device for observing said photochemical reaction on said microfluidic chip.

4. The microfluidic chip automatic system according to claim 1, wherein said gas source is a cylinder containing a high pressure gas or an air compressor.

5. The microfluidic chip automatic system according to claim 1, wherein a flowmeter is connected with said gas source for controlling an output flow rate of said gas source.

6. The microfluidic chip automatic system according to claim 1, wherein said controller further comprises a circuit board and a digital interface card, wherein said plural solenoid valves are connected with said digital interface card through said circuit board, and said digital interface card is further connected with said computer.

7. The microfluidic chip automatic system according to claim 1, wherein said controller further comprises a manifold device, wherein said manifold device comprises a main body and plural outlets.

8. The microfluidic chip automatic system according to claim 7, wherein said manifold device is connected with an additional gas source, wherein said reagent of said fluid source is introduced into said microfluidic chip through plural fluid pipes.

9. The microfluidic chip automatic system according to claim 1, wherein said high pressure gas is controlled by said plural solenoid valves to be introduced into said microfluidic chip through plural gas pipes.

10. The microfluidic chip automatic system according to claim 1, wherein said plural solenoid valves are 3 port solenoid valves.

11. The microfluidic chip automatic system according to claim 1, wherein said light source is a mercury lamp.

12. The microfluidic chip automatic system according to claim 1, wherein said light beam provided by said light source is a UV light beam.

13. The microfluidic chip automatic system according to claim 1, wherein said plural lenses comprises a first lens group, a second lens, and a third lens.

14. The microfluidic chip automatic system according to claim 13, wherein said light beam provided by said light source is transmitted through said first lens group, said digital micromirror device, said grating device, said second lens, said reflective mirror and said third lens sequentially.

15. The microfluidic chip automatic system according to claim 13, wherein said first lens group comprises three lenses.

16. The microfluidic chip automatic system according to claim 13, wherein said third lens is a focusing lens.

17. The microfluidic chip automatic system according to claim 1, wherein said computer has a designed image for controlling said position of said microfluidic chip to carry out said photochemical reaction.

18. The microfluidic chip automatic system according to claim 1, wherein said grating device further comprises an adjustable grating window.
